(19) United States
(12) Patent Application Publication
(10) Pub. No.: US 2024/0416135 A1
(43) Pub. Date: Dec. 19, 2024

(54) Integrated Header-Based Data and Charging Antenna for an Implantable Medical Device

(71) Applicant: Boston Scientific Neuromodulation Corporation, Valencia, CA (US)

(72) Inventors: Javad Paknahad, Pasadena, CA (US); Mizanur Rahman, Valencia, CA (US); Damon Moazen, Northridge, CA (US); John Rivera, Oxnard, CA (US)

(21) Appl. No.: 18/658,543

(22) Filed: May 8, 2024

Related U.S. Application Data
(60) Provisional application No. 63/507,924, filed on Jun. 13, 2023.

(51) Int. Cl. A61N 1/378; A61N 1/372; H02J 50/10

(52) U.S. Cl. CPC A61N 1/3787; A61N 1/37229; H02J 50/10

(57) ABSTRACT

An implantable medical device is disclosed having a single antenna structure in its header capable of receiving power from an external charger by near-field magnetic induction, and capable of communicating data with an external communication system by far-field radio frequency (RF) waves. The antenna structure preferably comprises a stamped conductive sheet and is generally loop shaped. The antenna in one example includes end connections and a center connection which acts as an RF feed, although the antenna may also have just two connections. An algorithm operable at least in the IMD can if necessary time multiplex the data and charging operations of the antenna and can configure the IMD's circuitry to operate in either a charging mode or a data communications mode.

### CROSS REFERENCE TO RELATED APPLICATIONS

This is a non-provisional of U.S. Provisional Patent Application Ser. No. 63/507,924, filed Jun. 13, 2023, which is incorporated herein by reference, and to which priority is claimed.

### FIELD OF THE INVENTION

The present invention relates generally to medical devices, and more particularly to antenna structures and circuitries useable in such devices.

### INTRODUCTION

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device (IMD) system.

As shown in FIG. 1, an SCS system typically includes an implantable pulse generator (more generally, an IMD) 10, which includes a biocompatible device case 12 formed of a conductive material such as titanium for example. The case 12 typically holds the circuitry and power source (e.g., a battery, not shown) necessary for the IMD 10 to function, although IMDs can also be powered continually via external and external charger and therefore may lack a battery. The IMD 10 is coupled to electrodes 16 via one or more electrode leads 18, such that the electrodes 16 form an electrode array 20. The electrodes 16 are carried on a flexible body 22, which also houses the individual signal wires 24 connected to each electrode. In the illustrated embodiment, there are eight electrodes (Ei) on two leads 18 for a total of sixteen electrodes 16, although the number of leads and electrodes is application specific and therefore can vary. The conductive case 12 may also comprise an electrode. The leads 18 connect to the IMD 10 using lead connectors 26, which are fixed in a non-conductive header material 28, which can comprise a non-conductive epoxy for example. Contacts at the proximal ends of the leads 18 connect to contacts in the lead connectors 26, which are in turn connected to feedthrough wires 29 that pass through a hermetic feedthrough 31 positioned between the header 28 and the case 12. The feedthrough wires 29 connect to circuitry (e.g., a circuit board) inside the case 12. The header 28, while shown at the top of the IMD, could be located anywhere with respect to the case 12.

An IMD 10 may include different antennas. For example, IMD 10 may include one or more telemetry antennas 34a and/or 34b used to wirelessly transmit/receive data to/from an external communication system 100, such as an external controller 60, a clinician programmer 70, or a system 80 as shown in FIG. 2. The IMD 10 may also include a charging antenna 36 for wirelessly receiving power from an external charger 90 to power the IMD or to charge its battery. IMD 10 is shown as having two telemetry antennas 34a and 34b, although typically an IMD 10 will have only one of these antennas, and in a particular an antenna that is compliant with the format of the antennas in the external communication system 100 with which they communicate.

Telemetry antenna 34a comprises a coil such as a winding of copper (e.g., Litz) wire, and communicates data with external communication systems 100 via a near-field bi-directional magnetic induction (MI) data link 104a. The telemetry antenna 34a in FIG. 1 is shown within the case 12 of the IMD 10, but it may also appear in the header 28 with the lead connectors 26. The telemetry antenna 34a can communicate with the external communication systems 100 along MI data link 104a via a protocol such as frequency shift keying (FSK), using modulation frequencies centered around 125 kHz, as described in U.S. Patent Application Publication 2015/0080982. This is just one example, and other data modulations schemes could be used as well. Telemetry antenna 34a may communicate via MI data link 104a (and inductively couple) with similar coils 64a, 74a, and 84a, in the controller 60, programmer 70, and system 80 respectively. As is known, communication along MI data link 104a involves energizing one of the coils (e.g., 64a) with modulated data, thus forming the MI data link 104a as a modulated magnetic field, which is received as an induced current at another coil (e.g., 34a), where it can then be demodulated to recover the data. Because magnetic induction operates to communicate at shorter distances, the effective communication distance of MI data link 104a (i.e., the distance between the IMD 10 and the external system) may comprise up to about 2 feet.

Telemetry antenna 34b in the IMD 10, by contrast, comprises a far-field RF antenna which is used to communicate with external systems via far-field electromagnetic waves via a bi-directional RF data link 104b. Telemetry antenna 34b may comprise a monopole or dipole, and may be formed as a wire, slot, or patch antenna. The telemetry antenna 34b in FIG. 1 is shown within the header 28 of the IMD 10, but it may also appear in the case 12. Telemetry antenna 34b and RF data link 104b may operate in accordance with a short-range RF communication protocol such as Bluetooth, Bluetooth Low Energy (BLE), WiFi, MICS, Zigbee, etc., as described in U.S. Patent Application Publication 2016/0051825. Such communications on RF data link 104b may occur generally at frequencies of 10 MHz to 10 GHz (e.g., 2.4 GHz in the case of Bluetooth). Telemetry antenna 34b may communicate via RF data link 104b with similar antennas 64b, 74b, and 84b, in the controller 60, programmer 70, and system 80 respectively. Far field communications on RF data link 104b operate at longer distances than do communications by magnetic induction, and so the effective communication distance of RF data link 104b (i.e., the distance between the IMD 10 and the external system) may comprise up to about 25 feet.

Charging antenna 36 in the IMD 10 receives wireless power via a magnetic induction power link 106 provided from an external charger 90. Like the telemetry antenna 34a, charging coil 36 comprises a coil such as a winding of copper (e.g., Litz) wire. The charging antenna 36 is shown in FIG. 1 within the case 12 of the IMD 10, but it may also appear in the header 28. When charging or powering the IMD, a primary charging coil 96 in the charger 90 is energized, creating a magnetic charging field along MI power link 106 which is received by the changing antenna 36 in the IMD 10, where the received power is rectified and used to power the IMD 10 or charge its battery. (Link 106 may also comprise a data-modulated magnetic field, thus capable of supplying data to the IMD 10 as well as power). The magnetic charging field provided by MI power link 106 may be at approximately 80 kHz in one example. Again, because magnetic induction operates at shorter distances, the effective communication distance of MI power link 106 may comprise up to about 2 feet, although typically the primary charging coil 96 is brought much closer to the IMD 10 (e.g., within 1-3 cm) to increase the charging efficiency and to speed charging of the IMD. In the example shown in FIG. 2, the charger 90's charging coil 96 is positioned within a charging head 94 that is coupled to an electronics module 92 by a cable, as described in U.S. Pat. No. 10,603,501. However, the charging coil 96 and related electronics may also be integrated in a single housing, as disclosed for example in U.S. Pat. No. 7,979,126.

As already introduced, FIG. 2 shows various external communication systems 100 that can wirelessly communicate data with the IMD 10, and an external charger 90 to power or charge the IMD 10. External communication systems 100 can be used to wirelessly transmit a stimulation program to the IMD 10—that is, to program its stimulation circuitry to produce stimulation at the electrodes 16 with desired amplitudes and timings. Such systems may also be used to adjust one or more stimulation parameters of a stimulation program that the IMD 10 is currently executing, and/or to wirelessly receive information from the IMD 10, such as various status information, etc. Although the external charger 90 used to power/charge the IMD 10 is shown in FIG. 2 separately from the external communication systems 100 used to communicate data with the IMD 10, in other examples, such powering/charging and data communications can be integrated in a single external device or system. See, e.g., U.S. Pat. Nos. 8,498,716 and 8,335,569.

External controller 60 may be as described in U.S. Patent Application Publication 2015/0080982 for example, and may comprise a portable, hand-held controller dedicated to work with the IMD 10. External controller 60 may also comprise a general-purpose mobile electronics device such as a mobile phone which has been programmed with a Medical Device Application (MDA) allowing it to work as a wireless controller for the IMD 10, as described in U.S. Patent Application Publication 2015/0231402, which is incorporated herein by reference. As already described, external controller 60 may include an MI antenna 64a and/or an RF antenna 64b capable of communicating with the IMD 10 along MI data link 104a and/or RF data link 104b. The external controller 60 is typically designed for patient use and like other external systems enables a patient to adjust stimulation parameters and to perform other forms of control and monitoring of the IMD 10, although it may have limited functionality when compared to systems 70 and 80 typically used by clinicians, described next.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 2, the computing device is shown as a laptop computer that includes typical computer user interface means (e.g., a display, buttons, mouse, keyboard, speakers, stylus, printer, etc.), not all of which are shown for convenience. Also shown in FIG. 2 are accessory devices for the clinician programmer 70 that are usually specific to its operation as an IMD controller. For example, a communication “wand” 76 coupleable to suitable ports on the computing device is shown. Typically, this wand 76 will include a MI antenna 74a capable of communicating with the MI antenna 34a in the IMD 10, where the wand 76 can be positioned in close proximity to the IMD 10 to allow communications to occur along MI data link 104a. The clinician programmer 70 (or the wand 76) may also include one or more RF antennas 34b to communicate with the RF antenna 34b in the IMD 10 via RF data link 104b at longer distances. The clinician programmer 70 can also communicate with other devices and networks, such as the Internet, either wirelessly or via a wired link provided at an Ethernet or network port.

External system 80 comprises another means of communicating with and controlling the IMD 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IMD's antenna 34a and/or 34b, such as a near-field MI coil antenna 84a and/or a far-field RF antenna 84b. Intermediary device 82 may be located generally proximate to the IMD 10 (consistent with the distances at which the data links 104a and 104b are effective). Network 85 can be accessed by any user terminal 87, which typically comprises a computer device associated with a display and associated computer peripherals. External system 80 allows a remote user at terminal 87 to communicate with and control the IMD 10 via the intermediary device 82.

U.S. Patent Application Publication 2023/0173273, which is incorporated herein by reference, describes external systems 60, 70, and 80 and IMD 10 in more detail, as well as describes circuitry inherent in these systems.

### SUMMARY

In a first example, an implantable medical device (IMD) is disclosed, which may comprise: a case; a non-conductive header attached to the case; an antenna within or on the header, wherein the antenna is configurable to operate in a first mode for far-field radiofrequency (RF) data communications with an external system, and in a second mode to receive a near-field magnetic charging field to power the IMD; and control circuitry within the case configured to configure operation of the antenna in the first and second modes in a time multiplexed manner.

In one example, the antenna is within the header. In one example, the header is overmolded over the antenna. In one example, the header further comprises at least one lead connector into which at least one lead can be inserted. In one example, the antenna is formed in a plane. In one example, the plane is perpendicular to a top of the case to which the header is attached. In one example, the plane is offset in the header in a direction parallel with the top of the case. In one example, the antenna comprises at least one planar sheet of metal. In one example, the antenna is coated, plated, or cladded with a conductive material. In one example, the antenna is three dimensional. In one example, the antenna does not comprise a wire. In one example, the antenna is loop shaped. In one example, the antenna does not comprise a continuous loop. In one example, the antenna comprises a top horizontal portion, a right vertical portion, and a left vertical portion. In one example, the antenna further comprises at least one bottom horizontal portion. In one example, the IMD further comprises a feedthrough between the header and the case. In one example, the IMD further comprises a plurality of electrical connections to the antenna, wherein the plurality of electrical connection pass through the feedthrough. In one example, a first and second of the electrical connections connect to first and second ends of the antenna. In one example, the IMD further comprises a resonant capacitor, wherein an inductance between the first and second ends and the resonant capacitor comprise a resonant tank to form an AC voltage across the first and second ends in the second mode in response to the magnetic charging field, wherein the AC voltage provides the power to the IMD. In one example, during the first mode the control circuitry configures a third of the electrical connections to operate as an RF feed for the RF data communications. In one example, during the first mode the first and second electrical connections capacitively couple to the case acting as a ground plane. In one example, none of the first, second, or third electrical connections are directly connected to the case in either of the first or second modes. In one example, during the second mode the third electrical connection is inactive. In one example, the control circuitry is configured to configure operation of the antenna in the first and second modes by configuring charging circuitry and/or telemetry circuitry in the case. In one example, the control circuitry is configured to control operation of the antenna in the first and second modes via a time multiplexing algorithm programmed in the control circuitry. In one example, the control circuitry is configured to default to operation in the first mode. In one example, the control circuitry is configured to receive a charging request to switch to operation in the second mode. In one example, the antenna is configured to receive the charging request. In one example, the IMD further comprises a charging sensor, wherein the charging request is provided from the charging sensor. In one example, the control circuitry is configured to default to operation in the second mode. In one example, the control circuitry is configured to automatically switch operation to the first mode after a duration. In one example, the IMD further comprises a battery, wherein in the second mode the received magnetic charging field is used to power the IMD by charging the battery. In one example, the radiofrequency (RF) data communications in the first mode comprise Bluetooth short-range communications.

In a second example, an implantable medical device (IMD) is disclosed which may comprise: an antenna having a first connection to a first end of the antenna, a second connection to a second end of the antenna, and a third connection to the antenna; wherein the antenna is operable in a first mode for far-field radiofrequency (RF) data communications with an external system using the third connection as an RF feed, wherein the antenna is operable in a second mode to receive a magnetic charging field to power the IMD, whereby an AC voltage is induced to form between the first and second connections via near-field magnetic induction to provide power to the IMD.

In one example, the IMD further comprises control circuitry configured to configure operation of the antenna in the first and second modes. In one example, the IMD further comprises a case, wherein the control circuitry is within the case. In one example, none of the first, second, or third electrical connections are directly connected to the case in either of the first or second modes. In one example, during the first mode the first and second electrical connections capacitively couple to a ground plane comprising the case. In one example, the IMD further comprises a non-conductive header attached to the case. In one example, the antenna is within or on the header. In one example, the header is overmolded over the antenna. In one example, the header further comprises at least one lead connector into which at least one lead can be inserted. In one example, the antenna is formed in a plane. In one example, the plane is perpendicular to a top of the case to which the header is attached. In one example, the plane is offset in the header in a direction parallel with the top of the case. In one example, the antenna is three dimensional. In one example, the IMD further comprises a feedthrough between the header and the case. In one example, the first, second, and third electrical connections pass through the feedthrough. In one example, in the first mode the antenna is configured as a first monopole antenna between the third connection and the first connection, and second monopole antenna between the third connection and the second connection. In one example, the antenna comprises at least one planar sheet of metal. In one example, the antenna is coated, plated, or cladded with a conductive material. In one example, the antenna does not comprise a wire. In one example, the antenna is loop shaped. In one example, the third connection is connected to a middle of the antenna between the first and second ends. In one example, the antenna does not comprise a continuous loop. In one example, the antenna comprises a top horizontal portion, a right vertical portion, and a left vertical portion. In one example, the antenna further comprises at least one bottom horizontal portion. In one example, the IMD further comprises a resonant capacitor, wherein an inductance between the first and second ends and the resonant capacitor comprise a resonant tank in the second mode. In one example, the third connection to the antenna is between the first and second connections. In one example, during the second mode the third electrical connection is inactive. In one example, the control circuitry is configured to configure operation of the antenna in the first and second modes by configuring charging circuitry and/or telemetry circuitry of the IMD. In one example, the control circuitry is configured to configure operation of the antenna in the first and second modes in a time multiplexed manner. In one example, the control circuitry is configured to control operation of the antenna in the first and second modes via a time multiplexing algorithm programmed in the control circuitry. In one example, the control circuitry is configured to default to operation in the first mode. In one example, the control circuitry is configured to receive a charging request to switch to operation in the second mode. In one example, the antenna is configured to receive the charging request. In one example, the IMD further comprises a charging sensor, wherein the charging request is provided from the charging sensor. In one example, the control circuitry is configured to default to operation in the second mode. In one example, the control circuitry is configured to automatically switch operation to the first mode after a duration. In one example, the IMD further comprising a battery, wherein in the second mode the received magnetic charging field is used to power the IMD by charging the battery. In one example, the radiofrequency (RF) data communications in the first mode comprise Bluetooth short-range communications.

In a third example, an implantable medical device (IMD) is disclosed, which may comprise: an antenna having only a first connection to a first end of the antenna and a second connection to a second end of the antenna; wherein the antenna is configurable to operate in a first mode for far-field radiofrequency (RF) data communications with an external system using the first connection as an RF feed, wherein the antenna is configurable to operate in a second mode to receive a magnetic charging field to power the IMD, whereby an AC voltage is induced to form between the first and second connections via near-field magnetic induction to provide power to the IMD; and control circuitry configured to configure operation of the antenna in the first and second modes.

In one example, the IMD further comprising a case, wherein the control circuitry is within the case. In one example, neither of the first or second electrical connections are directly connected to the case in either of the first or second modes. In one example, during the first mode the second electrical connection capacitively couples to a ground plane comprising the case. In one example, the IMD further comprises a non-conductive header attached to the case. In one example, the antenna is within or on the header. In one example, the header is overmolded over the antenna. In one example, the header further comprises at least one lead connector into which at least one lead can be inserted. In one example, the antenna is formed in a plane. In one example, the plane is perpendicular to a top of the case to which the header is attached. In one example, the plane is offset in the header in a direction parallel with the top of the case. In one example, the antenna is three dimensional. In one example, the further comprises a feedthrough between the header and the case. In one example, the first and second electrical connections pass through the feedthrough. In one example, the antenna comprises at least one planar sheet of metal. In one example, the antenna is coated, plated, or cladded with a conductive material. In one example, the antenna does not comprise a wire. In one example, the antenna is loop shaped. In one example, the antenna does not comprise a continuous loop. In one example, the antenna comprises a top horizontal portion, a right vertical portion, and a left vertical portion. In one example, the antenna further comprises at least one bottom horizontal portion. In one example, the IMD further comprises a resonant capacitor, wherein an inductance between the first and second ends and the resonant capacitor comprise a resonant tank in the second mode. In one example, the first electrical connection is coupled to telemetry circuitry during the first mode. In one example, during the second mode the first electrical connection does not comprise the RF feed. In one example, the control circuitry is configured to configure operation of the antenna in the first and second modes by configuring charging circuitry and/or telemetry circuitry in the case. In one example, the control circuitry is configured to configure operation of the antenna in the first and second modes in a time multiplexed manner. In one example, the control circuitry is configured to control operation of the antenna in the first and second modes via a time multiplexing algorithm programmed in the control circuitry. In one example, the control circuitry is configured to default to operation in the first mode. In one example, the control circuitry is configured to receive a charging request to switch to operation in the second mode. In one example, the antenna is configured to receive the charging request. In one example, the IMD further comprises a charging sensor, wherein the charging request is provided from the charging sensor. In one example, the control circuitry is configured to default to operation in the second mode. In one example, the control circuitry is configured to automatically switch operation to the first mode after a duration. In one example, the IMD further comprises a battery, wherein in the second mode the received magnetic charging field is used to power the IMD by charging the battery. In one example, the radiofrequency (RF) data communications in the first mode comprise Bluetooth short-range communications.

In a fourth example, an implantable medical device (IMD) is disclosed, which may comprise: an antenna comprising at least one planar sheet of metal, and having a plurality of connections comprising a first connection to a first end of the antenna and a second connection to a second end of the antenna; wherein the antenna is operable in a first mode for far-field radiofrequency (RF) data communications with an external system using one of the plurality of connections as an RF feed, wherein the antenna is operable in a second mode to receive a magnetic charging field to power the IMD, whereby an AC voltage is induced to form between the first and second connections via near-field magnetic induction to provide power to the IMD.

In one example, the IMD further comprises control circuitry configured to configure operation of the antenna in the first and second modes. In one example, the IMD further comprises a case, wherein the control circuitry is within the case. In one example, none of the plurality of electrical connections are directly connected to the case in either of the first or second modes. In one example, the IMD further comprises a non-conductive header attached to the case. In one example, the antenna is within or on the header. In one example, the header is overmolded over the antenna. In one example, the header further comprises at least one lead connector into which at least one lead can be inserted. In one example, the antenna is formed in a plane. In one example, the plane is perpendicular to a top of the case to which the header is attached. In one example, the plane is offset in the header in a direction parallel with the top of the case. In one example, the antenna is three dimensional. In one example, the IMD further comprises a feedthrough between the header and the case. In one example, the plurality of electrical connections pass through the feedthrough. In one example, the antenna is coated, plated, or cladded with a conductive material. In one example, the antenna is loop shaped. In one example, the antenna does not comprise a continuous loop. In one example, the antenna comprises a top horizontal portion, a right vertical portion, and a left vertical portion. In one example, the antenna further comprises at least one bottom horizontal portion. In one example, the IMD further comprises a resonant capacitor, wherein an inductance between the first and second ends and the resonant capacitor comprise a resonant tank in the second mode. In one example, the plurality of connections further comprise a third connection to the antenna. In one example, the third electrical connection comprises the RF feed in the first mode. In one example, during the second mode the third electrical connection is inactive. In one example, the first electrical connection comprises the RF feed in the first mode. In one example, during the second mode the first electrical connection is does not comprise the RF feed. In one example, the control circuitry is configured to configure operation of the antenna in the first and second modes by configuring charging circuitry and/or telemetry circuitry in the case. In one example, the control circuitry is configured to configure operation of the antenna in the first and second modes in a time multiplexed manner. In one example, the control circuitry is configured to control operation of the antenna in the first and second modes via a time multiplexing algorithm programmed in the control circuitry. In one example, the control circuitry is configured to default to operation in the first mode. In one example, the control circuitry is configured to receive a charging request to switch to operation in the second mode. In one example, the antenna is configured to receive the charging request. In one example, the IMD further comprises a charging sensor, wherein the charging request is provided from the charging sensor. In one example, the control circuitry is configured to default to operation in the second mode. In one example, the control circuitry is configured to automatically switch operation to the first mode after a duration. In one example, the IMD further comprises a battery, wherein in the second mode the received magnetic charging field is used to power the IMD by charging the battery. In one example, the radiofrequency (RF) data communications in the first mode comprise Bluetooth short-range communications.

### BRIEF DESCRIPTION OF THE DRAWINGS

FIG. 1 shows an Implantable Pulse Generator (a type of IMD), and the manner in which an electrode array is connected to the IMD, in accordance with the prior art.

FIG. 2 shows external communication systems and an external charger capable of communicating with and powering the IMD, in accordance with the prior art.

FIGS. 3A-3D show an example of a single antenna structure in the IMD's header to enable both data communications and charging of the IMD, in accordance with an example of the invention.

FIG. 4 shows the circuitry in the IMD, including charging circuitry and telemetry circuitry, and including a time multiplexing algorithm to configure the IMD for data communications or charging.

FIG. 5 shows external communication systems and an external charger capable of communicating with and powering the IMD of FIGS. 3A-4.

FIG. 6 shows a first example of the time multiplexing algorithm for controlling the IMD's operation in a data communications mode and a charging mode.

FIG. 7 shows a second example of the time multiplexing algorithm in which the modes are implemented as interleaved charging periods and telemetry periods.

FIG. 8 shows a third example of the time multiplexing algorithm in which operation defaults to the charging mode in the IMD.

FIGS. 9A-9D show examples in which the antenna structure includes various extensions to improve data communications performance.

FIGS. 10A-10E show examples in which the antenna structure can be made three dimensional in shape to improve data communications performance.

FIGS. 11A-11D show examples in which the antenna structure includes only two connection points.

FIG. 12 shows modifications to the circuitry when only two connection points are used.

FIG. 13 shows an inflatable penile implant (a type of IMD) that can incorporate any of the disclosed antenna structures, related circuitry, and algorithms.

### DETAILED DESCRIPTION

The inventors find it unfortunate that IMDs, like IMD 10 described earlier, typically requires two separate antennas: one (34a and/or 34b) for data communications (e.g., IMD control and/or monitoring) with external communication systems 100 (e.g., 60, 70, 80); and another (e.g., 36) to allow the IMD 10 to be powered or charged by an external charger 90. Having two separate antennas complicates the design and manufacturing of the IMD and increases its cost.

The inventors also find it unfortunate that at least some IMD designs place one or more of the antennas within the case 12 of the IMD 10. As noted earlier, this case 12 is typically conductive, which tends to attenuate the MI or RFs fields (MI data link 104a, RF data link 104b, MI power link 106) that pass to or from these antennas. This diminishes the distances at which these links can operate, or requires these links to operate at higher powers to compensate. Alternatively, placing antennas within the case 12 may require increasing the size (e.g., area) of the antennas to compensate for such attenuation and to increase signal strength. This is not preferred, because an IMD is preferably as small as possible to ease inconvenience to the patient.

It is preferable in the inventor's view to position the antennas in the IMD's header 28. The header 28 as discussed earlier typically comprises a non-conductive, dielectric material such as epoxy or plastic, which does not significantly attenuate MI or RF fields. This allows the antennas to be made smaller, reducing IMD size, and allows the power of supported communication links to be lower.

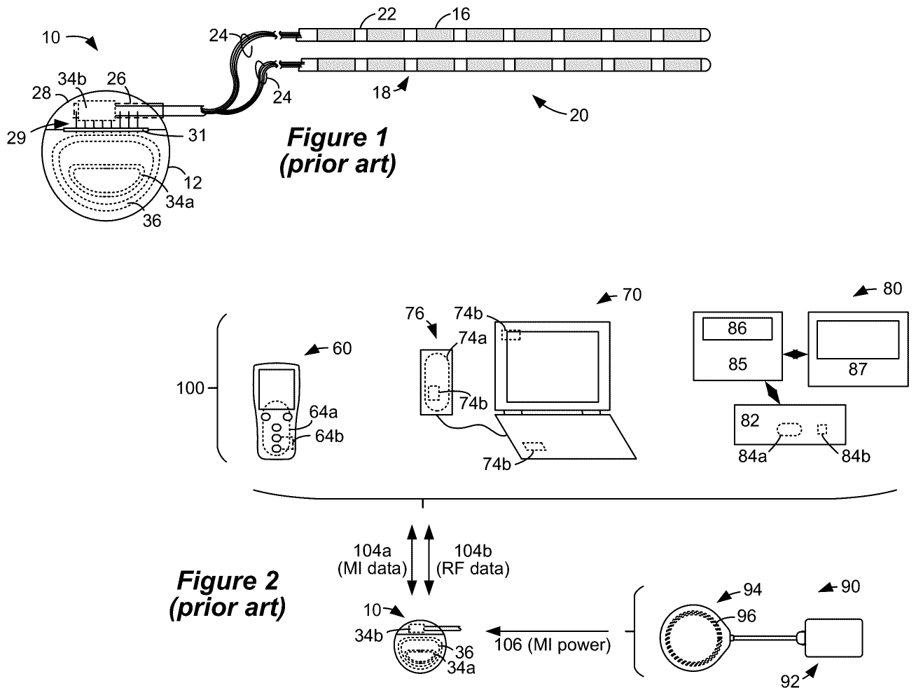

Placing antennas in the header 28 however has drawbacks. The header 28 is preferably small to reduce IMD size, and typically includes other structures that take significant volume, such as the lead connectors 26 (FIG. 1). As such, there is limited room in the header 28 to accompany an antenna, and this is especially true should the header 28 need to accompany a number of antennas, such as a RF antenna (e.g., 34a or 34b) for data communications (on data links 104a or 104b), and a charging antenna 36 to receive power (on MI power link 106). U.S. Pat. No. 8,929,986 provides an example in which separate data and charging antennas are provided in an IMD's header.

U.S. Pat. No. 9,750,930 provides a different example in which a single antenna is provided in an IMD header that provides both data and charging functionality. While this approach comprises an improvement in that it only uses a single antenna structure in the header, it requires the use of complicated filter circuitry to prevent data communications and charging from conflicting with each other, i.e., to prevent power received at the antenna from interfering with the data telemetry circuitry, and to prevent data transmission and reception from interfering with charging circuitry. The adequacy of the filter circuitry to address the interference between data communication and charging functionality could be potentially compromised due to the presence of a rectifier in the charging circuitry. In the GHz frequency range utilized for RF data communications in the '930 patent, the parasitic capacitances in the rectifier could unintentionally short such data communications. This could negatively affect the power transfer efficiency of RF data communications, potentially affecting the overall performance.

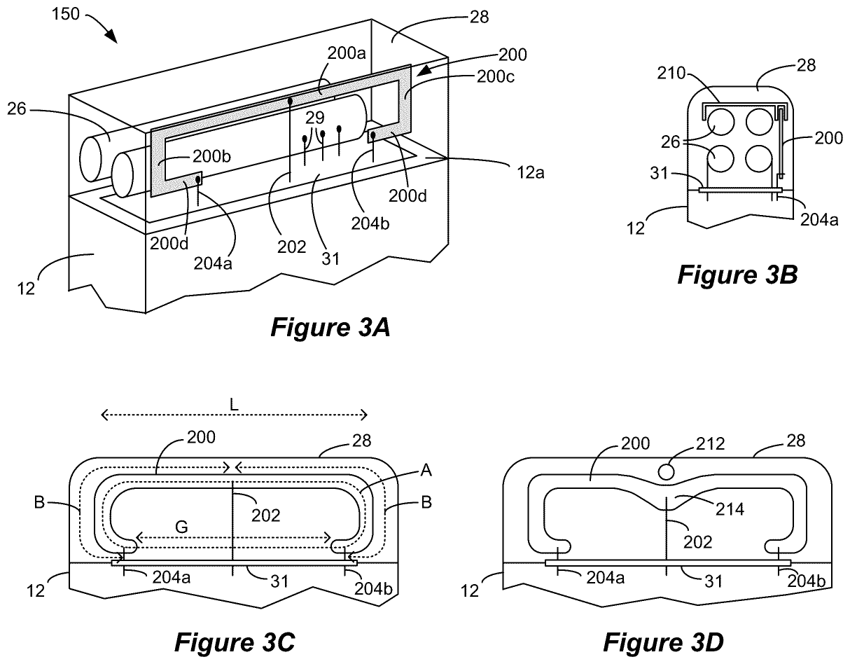

The inventors' antenna structure 200 for an IMD 150, and related circuitry, improves upon these prior approaches. As shown first in various views in FIGS. 3A-3D, the disclosed antenna structure 200 is operable both for the purpose of bi-directional data communications and for IMD charging/power reception. As explained in detail later, complicated filtering circuitry is not required to prevent interference between data and charging functionality. A time multiplexing scheme may be used in which the IMD 150 is controlled to enable data communications and charging at different times. Time multiplexing may be enabled by use of a time multiplexing algorithm 300 operable at least in the IMD 150, as described later. Such time-multiplexed use of the antenna structure 200 can comprise a better and more organized manner of handling potential conflicts between IMD data communications and power reception when compared to reliance on the use of filtering circuitry. Time multiplexed use of the disclosed antenna structure, while useful, is not strictly required, as explained further below.

As shown in FIG. 3A and the side view of FIG. 3B, the antenna structure 200 is positioned within the header 28 of the IMD 150 along with the lead connectors 26 mentioned previously, two of which are shown in FIG. 3A, and four of which are shown in the side view of FIG. 3B. As shown, the antenna structure 200 is preferably offset within the header 28 (to the right in FIG. 3B), in what would preferably be the outside-facing side of the IMD when implanted in the patient (closer to the external systems with which the IMD communicates). This is preferred to minimize electrical interference of communications with the antenna structure 200, i.e., to minimize interference with other conductive structures (e.g., the lead connectors 26) in the header 28. That being said, the antenna structure 200 can also occur at different locations in the header 28. For example, the plane of antenna structure 200 could be parallel with and proximate to the top or any of the four sides of the header 28. The antenna structure 200 can also be placed on the outside surface of the header. In other IMD designs not including leads, the header 28 may not include lead connectors 26, or may include other structures or ports.

Although not shown, the antenna structure 200 could also be provided within the case 12, but as discussed earlier this is less preferable if the case is conductive, as this will attenuate communications with the antenna. That being said, not all IMD cases 12 are conductive (e.g., some are ceramic) and thus would not significantly attenuate such communications.

The antenna structure 200 preferably does not comprise a wound coil, and is preferably not made of wire (e.g., of circular cross section), although it could be. Instead, the antenna structure 200 preferably comprises at least one planar sheet of metal formed (e.g., by stamping or milling) into the requisite shape. The antenna structure 200 can take on different shapes, as discussed subsequently. The antenna structure 200 may be made from any number of conductive materials or alloys, such as those containing titanium, copper, gold, silver, and the like. The antenna structure 200 may also include combinations of alloys formed in distinctive layers, and may be coated, plated, or cladded with conductive materials such as gold. Regardless of how it is fabricated, antenna structure 200 is preferably biocompatible. Because the antenna structure 200 is preferably planar and formed from a conductive sheet of material, it is thin, and as best shown in FIG. 3B beneficially does not take significant room in the header 28. Preferably the plane of the antenna structure 200 is perpendicular to the top 12a of the case to which the header 28 is attached. That being said, the antenna structure 200 can also be three-dimensional in other examples, as shown later. Although not shown, the antenna structure 200 could also be formed on a substrate, such as on a printed circuit board.

The antenna structure 200 is preferably fully encompassed within the header 28. For example, and like the lead connectors 26, the antenna structure 200 is preferably overmolded with the material (e.g., epoxy) used to form the header 28. To assist in such overmolding, and to otherwise mechanically stabilize the electrical components in the header 28 during the IMD 150's manufacture, the antenna structure 200 may be permanently or temporality stabilized using a support structure. For example, FIG. 3B shows use of a clip 210 to stabilize the antenna structure 200 within the header 28 before overmolding. This clip 210 preferably holds the antenna structure 200 to the lead connectors 26, which are relatively rigidly affixed to the IMD 150 by the feedthrough wires 29. A support structure for the antenna structure 200 could also rigidly affix the antenna structure to the case 12, to the top 12a of the case, or to other IMD structures. While useful, a support structure for the antenna 200 is not strictly required, as the electrical connections 202, 204a and 204b (described later) to the antenna structure 200 may provide sufficient mechanical stability. While use of overmolding is preferred to form the header 28, the header may also be separately pre-formed as a solid piece, which is then placed over structures within the header 28 (e.g., the lead connectors 26; the antenna structure 200; any support structures) and then affixed to the case 12, preferably in a medically hermetic manner.

Because the antenna structure 200 is within the non-conductive dielectric header 28, and not within the conductive case 12, communications (data, power) with the antenna structure are not significantly attenuated. Therefore, the antenna structure 200 can more easily (and with greater distance and/or lower power) transmit and send data and receive power from external systems, as explained subsequently.

The header 28 may be relatively rounded in shape to promote patient comfort, as best shown in FIGS. 3B-3D. The antenna structure 200 may likewise be relatively rounded in shape, e.g., to match the rounded profile of the header 28, as best shown in FIGS. 3C and 3D. Additionally, the shape of the antenna structure 200 may be modified to accommodate other aspects of the header 28. For example, in FIG. 3D, the IMD 150's header 28 includes a suture hole 212 allowing the IMD 150 to be affixed (by a suture, not shown) at a particular position in the patient's tissue. This suture hole 212 appears at what is essentially the midpoint of the header 28, and to accommodate this, the antenna structure 200 includes a dip 214 to route the antenna structure 150 below this suture hole 212. This is in comparison to the antenna structure 200 as shown in FIG. 3C, which has no suture hole 212. From an electrical and performance standpoint, the examples shown in FIGS. 3C and 3D are not materially different.

The antenna structure 200 as shown in FIG. 3A is generally loop shaped, comprising a top horizontal portion 200a, left and right vertical portions 200b and 200c, and one or more bottom horizontal portions 200d. These portions may not be perfectly straight, or perfectly horizonal or vertical. These portions may also not be perfectly complete (e.g., in the case of portions 200d). That is, the antenna structure 200 may not comprise a continuous loop, although it could, including a loop with multiple turns. In one example, the length L of top horizontal portion 200a is approximately in the range of 20-30 mm, while left and right vertical portions 200b and 200c have a length in the range of approximately 10-15 mm. The length of bottom horizontal portions 200d can be variable, and these portions may be of different lengths.

In the depicted example, the antenna structure 200 comprises three electrical connections 202, 204a, and 204b. The connections 202, 204a, and 204b, like the feedthroughs 29 described earlier, connect through the feedthrough 31 at the top 12a of the case 12 to relevant electrical circuitry within the case 12, as explained subsequently.

Connections 204a and 204b are preferably connected to ends of the antenna structure 200 (e.g., to the ends of portions 200d). During power reception, and similar to a traditional power reception coil, the antenna structure 200 is magnetically inductively coupled to the charger 270 via the MI power link 250, and comprises an area A (FIG. 3C) to capture a magnetic flux of that link. This area A may not be completely bounded by the portions of the antenna structure 200; for example, there may be a gap G (FIG. 3C) in the antenna structure between the portions 200d as shown. Gap G can also occur at other locations (portions 200a, b, or c) of the antenna structure 200. Nevertheless, the antenna structure 200 generally defines area A to capture magnetic flux provided by MI power link 250. Connection 202 is not implicated, and is disabled (e.g., floating), during power reception, as explained further below.

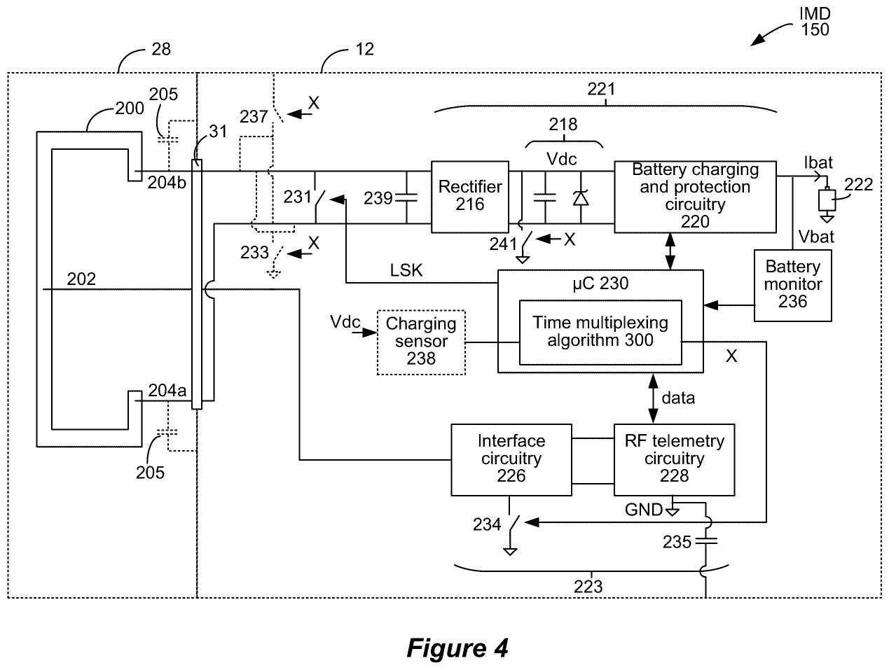

As shown in the circuit diagram FIG. 4, the connections 204a and 204b are connected in parallel with a resonant capacitor 239, although a series connection could be used as well. This resonant capacitor 239 is preferably within the case 12 of the IMD 150, but could be in the header 28 as well, as explained for example in U.S. Patent Application Publication 2022/0088396. Together, the antenna structure 200 and the resonant capacitor 239 comprise a resonant tank, which resonates at a frequency as set by the capacitance of the resonant capacitor (e.g., 8.5 nF) and an inductance of the antenna structure between connections 204a and 204b (e.g., in a range of 60-70 nH). Preferably, resonance is matched to the frequency of the MI power link 250 provided by the charger 270, which in one example comprises 6.78 MHz within the Industrial, Scientific, and Medical (ISM) radio band. Generally speaking,, the frequency used for power transmission along MI power link 250 can vary in range of about 20 kHz to 20 MHz. Charging at higher frequencies within this range is beneficial because it lowers heating in the case 12 by reducing the impact of eddy currents that form in the case 12 in response to the AC magnetic charging field on MI power link 250.

Power received via MI power link 250 builds an AC voltage between connections 204a and 204b at the ends of the antenna structure 200, which is rectified by rectifier circuitry 216 to establish a DC voltage, Vdc. As shown in circuitry 218, Vdc may be stabilized by a storage capacitor, and a Zener diode may be provided to ensure that Vdc does not exceed a particular threshold. Vdc can be provided to battery charging and protection circuitry 220, which can in turn control the charging of the battery 222, i.e., by providing a battery charging current Ibat and/or a battery voltage Vbat. As noted earlier, the IMD 150 may also lack a battery 222, and in this circumstance, Vdc can be used more generally to power the IMD 150. Battery monitoring circuitry 236 can monitor the charging process, such as by monitoring the voltage Vbat of the battery, the charging current Ibat provided to the battery, or other relevant charging information (e.g., temperature), and can report such charging information to the IMD's control circuitry 230, which is discussed further below. Circuits 216, 218, and 220 may collectively be referred to as charging circuitry 221.

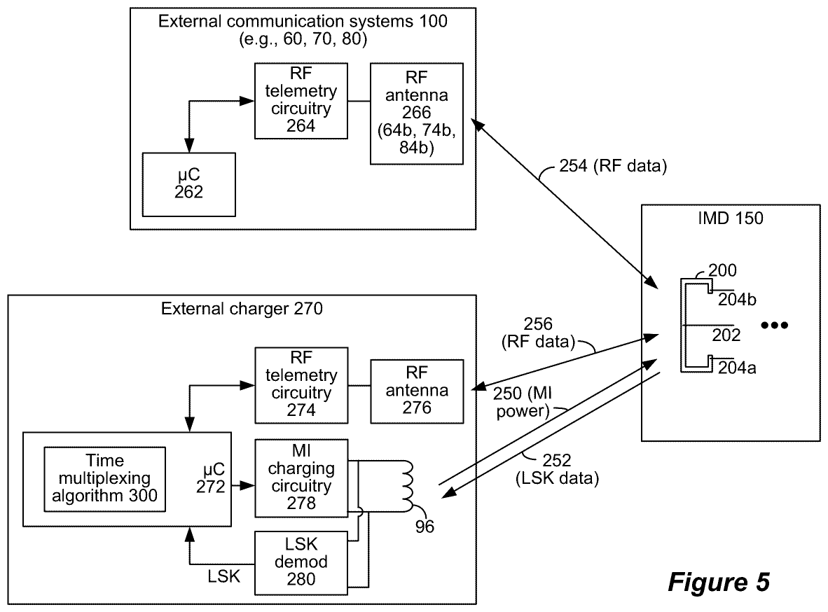

The IMD 150 may further include circuitry to communicate the status of the battery back to the charger 270 during power reception. While such communications can occur in different ways (including by use of the RF telemetry circuitry 228 described subsequently), the IMD 150 can also use Load Shift Keying to communicate such status information back to the charger, shown as LSK data link 252 in FIG. 5. Load Shift Keying to communicate with a charger is described for example in U.S. Patent Application Publication 2013/0096652. LSK involves modulating the impedance of the antenna structure 200 with data bits (“LSK”) provided by the control circuitry 230 to be serially transmitted from the IMD 150 to the external charger 270 along LSK data link 252. For example, and depending on the logic state of a bit to be transmitted, the ends of the antenna structure 200 can be selectively shorted to each other via a switch 231 to modulate the antenna structure's impedance. The impedance of the antenna structure 200 can be modulated in other ways.

At the external charger 270, and as shown in FIG. 5, an LSK demodulator 280 (FIG. 5) determines whether a logic ‘0’ or ‘1’ has been transmitted from the IMD 150 by assessing the magnitude of AC voltage that develops across the external charger's coil 96 as this coil is producing the magnetic charging field on MI power link 250. In effect, modulating the antenna structure 200 in the IMD 150 in this manner can be thought of as creating a reflection in the magnetic charging field along MI power link 250 that the charger 270 can assess to recover the LSK data. As shown, the demodulated data (LSK) can be reported to the external charger's control circuitry 272 for analysis. Such LSK back telemetry from the IMD 150 to the charger 270 can provide useful data concerning charging to the external charger 50, such as the capacity of the IMD's battery 14, or whether charging of the battery 14 is complete and operation of the external charger 270 and the production of magnetic charging field on MI power link 250 can cease. As discussed earlier, battery monitoring circuitry 236 can provide such charging information to the control circuitry 230.

Connection 202 is preferably connected at or proximate to the middle of the antenna structure 200, such as at the middle of top portion 202a, although this connection 202 could also appear at any location along the antenna between connections 204a and 204b. Connection 202 comprises an RF feed used during data transmission and reception along bidirectional RF links. As shown in FIG. 5, a number of such data RF links can be supported by the IMD 150, such an RF data link 254 with external communication systems 100 (e.g., the external controller 60, the clinician programmer 70, and/or the external system 80), and/or an RF data link 256 with the charger 270. RF data links 254 and 256 may be established in accordance with a short-range RF communication protocol, such as Bluetooth (at 2.4 GHz).

The physics involved in RF data transmission and reception using the antenna structure 200 is different than during charging. Whereas the antenna structure 200 operates as a loop to capture magnetic flux via magnetic induction along MI power link 250 during power reception, the antenna structure 200 instead operates as a far-field EM antenna during data communications, and thus does not work on a principle of magnetic induction. As explained further below, during data communications, the connections 204a and 204b may be left floating, using the case 12 (e.g., top 12a and feedthrough 31) as a ground plane. In this regard, connections 204a and 204b include parasitic capacitances 205 to the case 12 are shown in dotted lines in FIG. 4. The case 12 in turn, and as shown in FIG. 4, can be coupled to system ground (GND) by a low value capacitor 235 (e.g., 12 pF). Capacitances 205 and 235 essentially acts as short circuits at the higher frequencies (e.g., 2.4 GHz) at which data communications operate, thus allowing case 12 to operate as a ground plane at these frequencies. During receipt of the lower-frequency magnetic charging field via MI power link 250, the impedance of parasitic capacitances 205 would be high, and essentially would operate as open circuits.

In an alternative, during data communications, connections 204a and 204b may be actively coupled to system ground or the case 12 via optional switches 233 or 237 respectively, as shown in dotted lines. These switches 233 or 237 may be controlled via control signal X, which is explained further below.

RF feed 202 establishes antenna structure 200 as two monopole antennas operating in parallel: one between RF feed 202 and connection 204a, and one between RF feed 202 and connection 204b. In this example, each of these monopole antennas has the same length B (FIG. 3C), although these lengths could also be different, for example by not connecting connection 202 exactly at the middle of the top portion 200a, or by varying the lengths of portions 200a-200d discussed earlier. Lengths B would generally be between about 1-5 cm, which generally corresponds to one-quarter of a wavelength of the frequencies used on RF data links 254 and 256. For example, if Bluetooth is used for these links (e.g., 2.4 GHz), this would equate to about 2-3 cm, which is consistent with the lengths B. One skilled in the art will understand that length B may be adjusted for better performance at the frequency in question. For example, gap G (FIG. 3C) could be made longer or shorter to affect length B.

As shown in FIG. 4, the IMD 150 includes RF telemetry circuitry 228 operable in accordance with the short-range RF protocol being used, and may comprise a typical Bluetooth chip set. Interface circuitry 226 intervenes between the RF telemetry circuitry 228 and the RF feed 202. This interface circuitry 226 may comprise necessary matching networks and/or balun circuitry, as one skilled in the art will understand. As is typical, the RF telemetry circuitry 228 communicates digital data with the IMD's control circuitry 230. That is, the RF telemetry circuitry 228 provides demodulated digital data received from external systems (e.g., new or updated stimulation parameters for the IMD) to the IMD's control circuitry 230, and modulates data received from the control circuitry 230 (e.g., IMD status information) for transmission to external systems. RF telemetry circuitry 228 and related circuitry such as interface circuitry 226 are collectively referred to as telemetry circuitry 223.

Advantageously, the IMD's case 12 can still be used as a stimulation electrode during either power reception or data communications. The frequency at which stimulation occurs at the electrodes (e.g., 10 kHz or less) is significantly less than the frequencies involved in RF data links 254 and 256 (e.g., 2.4 GHz) and MI power link 250 (e.g., 6.78 MHz). At these lower frequencies, capacitor 235 between system ground and the case 12 will effectively act as an open circuit. As such, the case 12 can continue to be used as a stimulation electrode without shorting the case 12 to system ground.

As noted earlier, data and charging access to the antenna structure 200 may be time multiplexed. Such time multiplexing is preferably controlled by a time multiplexing algorithm 300 programmed (e.g., as firmware) in the IMD's control circuitry 230. The time multiplexing algorithm 300 can also operate in some examples, at least in part, in the external charger 270 (as firmware in its control circuitry 272), as explained further below with reference to FIG. 7. Control circuitries 230 and 272 may comprise a microcontroller for example, such as Part Number MSP430, manufactured by Texas Instruments, which is described in data sheets accessible on the Internet. Other types of control circuitry may be used in lieu of a microcontroller as well, such as microprocessors, FPGAs, DSPs, or combinations of these, etc. Control circuitries 230 and 272 may also be formed in whole or in part in one or more Application Specific Integrated Circuits (ASICs), such as in described in Application Specific Integrated Circuits (ASICs), as described in U.S. Patent Application Publications 2012/0095529, 2012/0092031, and 2012/0095519, which are incorporated by reference. One skilled in the art will understand that these control circuitries may provide general functionality in the IMD 150 and the charger 270 beyond operation of the time multiplexing algorithm 300. For example, control circuitry 230 may include or interface with stimulation circuitry in the IMD 150 that provides stimulation to the electrodes, as described earlier.

The time multiplexing algorithm 300 determines whether the antenna structure 200 of the IMD 150 can be used for data communication or for power reception at any given time, and issues a control signal X accordingly (e.g., X=1 during data communications, X=0 during power reception). In one example, the algorithm 300 can default to data reception (X=1), meaning that the algorithm 300 will generally configure the IMD's circuitry to permit the antenna structure 200 to be used for data communications. When charging is needed or requested, the algorithm 300 can instead configure the circuitry for power reception (X=0). As explained further below, control signal X can selectively enable or disable the charging circuitry 221 or the telemetry circuitry 223 in different ways. While it is preferred that the algorithm 300 set data communications as a default mode, it may also set charging as a default mode, as explained further below.

The status of control signal X can be set by the time multiplexing algorithm 300 in different ways. In one example, a request to charge the IMD 150 can be transmitted to the IMD 150 as data by an external system, such as the charger 270 (FIG. 5), thus allowing the algorithm 300 to configure the IMD's circuitry for charging (X=0). Alternatively, the IMD 150 can detect the presence of a magnetic charging field along MI power link 250 that has already been established by the charger 270. For this option, the IMD 150 includes a charging sensor 238, which can operate to detect the presence of a magnetic charging field on MI power link 250. In one example, the charging sensor 238 can comprise a magnetic field detector (e.g., a Hall or Reed sensor), which can report the detection of the magnetic charging field to the algorithm 300. In another example, the charging sensor 238 can receive information from the charging circuitry 221 indicative that a magnetic charging field is being received. For example, receipt of the charging field may cause rectifier circuitry 216 to produce a significant voltage Vdc, and so this voltage can be reported to the charging sensor 238. In any event, the charging sensor 238 can report the presence of the magnetic charging field to the algorithm 300, which can set control signal X (X=0) for charging.

When the algorithm 300 configures the IMD 150 for power reception (X=0), the charging circuitry 221 in the IMD may be enabled and the telemetry circuitry 223 may be disabled. In the depicted example, the charging circuitry 221 can be enabled by control signal X opening a switch 241 coupled between Vdc ground, thus allowing Vdc to form. The battery charging and protection circuitry 220 may also be specifically enabled by control signal X, although this isn't shown. Switches 233 and 237, if present, would also be opened. Connections 204a and 204b preferably float during power reception, which is desired for proper resonance, and ultimately to build Vdc to a suitable value as described earlier. The telemetry circuitry 223 may also disabled. In the depicted example, this occurs by control signal X opening a switch 234 coupled to ground, which disables the interface circuitry 226. Control signal X can also be used to enable or disable the RF telemetry circuitry 228 as well, although this isn't shown. Disabling the telemetry circuitry 223 in this manner allows RF feed connection 202 to float during power reception.

When the algorithm 300 configures the IMD 150 for data communications (X=1), the charging circuitry 221 in the IMD may be disabled and the telemetry circuitry 223 may be enabled. In the depicted example, the control signal X can close switch 241, which grounds Vdc to prevent charging. Although not shown, control signal X may also disable the charging and protection circuitry 220. Switches 233 and 237, if present, may also be closed, thus disabling charging by preventing a resonant voltage from building between connections 204a and 204b should a magnetic charging field (along MI power link 250) be present. The telemetry circuitry 223 is enabled by closing switch 234, thus allowing the interface circuitry 226 and/or the RF telemetry circuitry 228 to operate, and to generate or receive an RF feed signal at connection 202. The depicted manner by which the algorithm 300 and control signal X can be used to selectively enable or disable the charging circuitry 221 or the telemetry circuitry 223 is just one example. Examples using other enablement or disablement mechanisms are possible.

FIG. 5 has largely been discussed, but shows the various communication links involved in communicating with and powering an IMD 150 having the antenna structure 200 and circuitry just described. When the IMD 150 is operating to receive power in a charging mode (X=0), MI power link 250 from the charger 270 is active, as is the LSK data link 252 to communicate charging information back to the charger. When the IMD 150 is operating in a data communications mode (X=1, preferably its default state), the IMD 150 can communicate on RF data links 254 with traditional external communication systems 100 (e.g., 60, 70, and/or 80). These external systems as shown will include RF telemetry circuitry 264 and an RF antenna 266 (e.g., 64b, 74b, 84b) compliant with the short-range RF protocol supported by the IMD 150, such as Bluetooth. Like the IMD 150, the RF telemetry circuitry 264 in these external systems may include interface circuitry between the telemetry circuitry 264 and the antenna 266, but this detail isn't shown. These external systems will also include control circuitry 262.

The IMD 150 can also communicate on RF data link 256 with the charger 270 in the data communications mode (X=1). In this regard, the charger 270 may similarly include RF telemetry circuitry 274 and an RF antenna 276 compliant with the short-range RF protocol supported by the IMD 150. It is preferred that charger 270 include RF telemetry circuitry 274 to communicate with the IMD 150 via RF data link 256, as this can facilitate operation of the time multiplexing algorithm 300, described next. That being said, this is not strictly required, and instead the IMD 150 can communicate with the charger 270 through different means (e.g., by modulating MI power link 250 and/or by LSK data link 252).

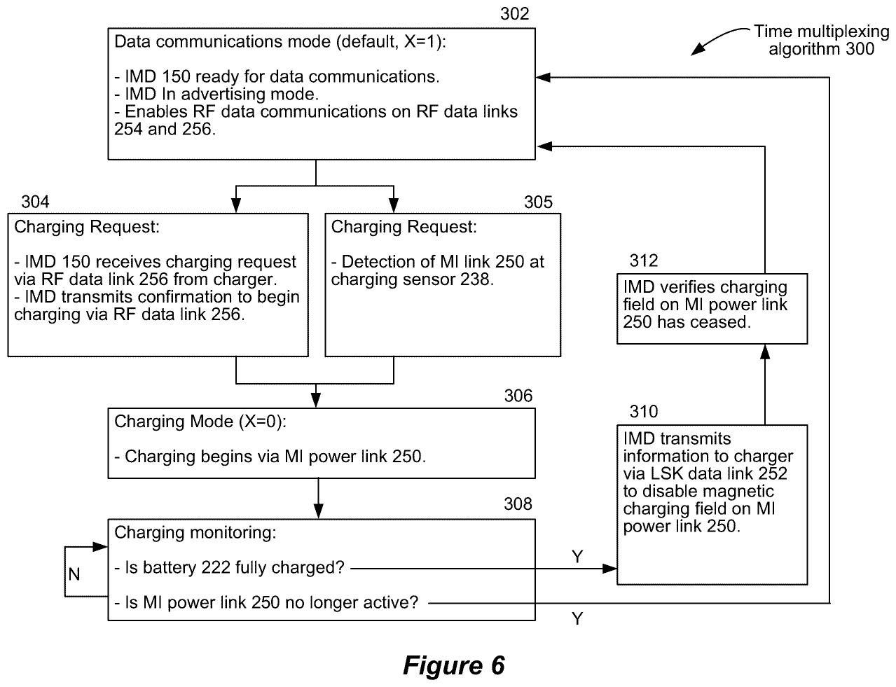

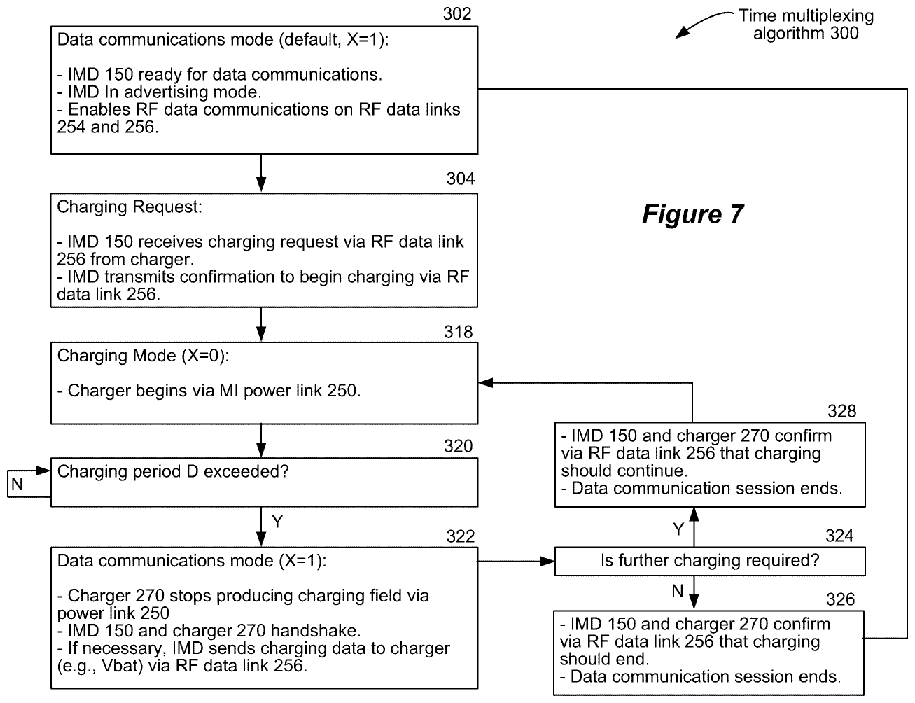

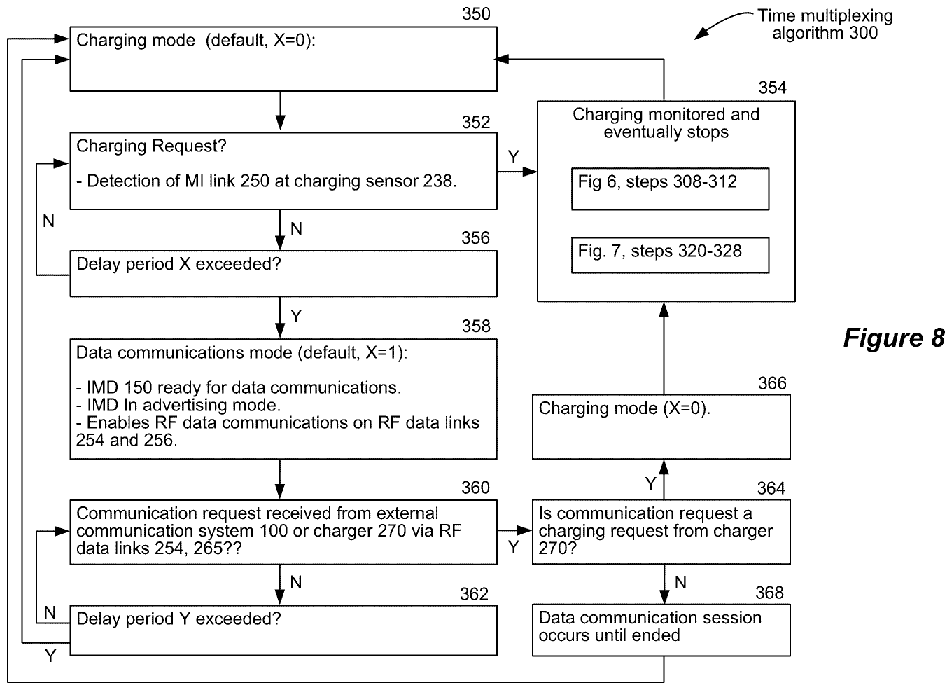

FIG. 6 describes a first example of steps involved during the operation of time multiplexing algorithm 300. As noted earlier, the algorithm 300 at step 302 defaults to use of a data communication mode (X=1) in which the IMD 150 configures the circuitry to enable the antenna structure 200 to be used for bidirectional RF communications on RF data links 254 and 256. In this mode, the various switches in the IMD can be closed or opened as described earlier to enable the telemetry circuity 223 and/or disable the charging circuitry 221. At this step, and assuming that a short-range protocol such as Bluetooth is used, the IMD 150's telemetry circuitry 228 may periodically broadcast advertising data on links 254 and 256 which external communication systems 100 and the external charger 270 can detect to establish a data communication session with the IMD. The use of advertising data in this context is discussed further in U.S. Pat. No. 11,576,223, which is incorporated by reference, and with which the reader is assumed familiar.

At this time, an external communication system 100, or the charger 270, can transmit a connection request to the IMD 150, which the IMD 150 can acknowledge to establish a communication session during which data can be telemetered between the devices. Colloquially, this means of establishing a communication session may be referred to as “handshaking” between the relevant external device and the IMD 150. While in the data communications mode, an external communication system 100 can for example send new or updated stimulation parameters for the IMD 150 to execute, or the IMD 150 can send status information to those systems. More relevantly to next steps in the algorithm 300, the charger 270 can also transmit a request to the IMD 150 to begin charging the IMD via MI power link 250, as discussed next.

The IMD 150 receives a request from the charger 270 to begin charging the IMD 150. This can occur in different manners as shown in steps 304 and 305. In the preferred example of step 304, the charger 270 can transmit via RF data link 256 a request to being charging. This request may be generated at the charger 270 without the user's knowledge. For example, the user of the charger 270 may simply turn on the charger 270, or otherwise select an input from a user interface of the charger 270 to begin charging. This may automatically cause (through operation of the charger's control circuitry 272), the charger 270 to automatically “handshake” with the IMD 150 via RF data link 256 to send the charging request. Alternatively, the sending of the charging request may be manual, with the user using the user interface of the charger 270 to transmit a charging request.

Once the charging request has been received at the IMD 150 at step 304, the algorithm 300 can determine whether charging can be enabled at the IMD 150, and hence whether the charger 270 can begin generating its magnetic charging field. Generally, the algorithm 300 would allow IMD charging to occur when requested, but may not do so (or may not do so immediately) if the algorithm 300 understands that the IMD 150 is not presently in a position to allow charging. For example, the algorithm 300 may understand the IMD 150 to be currently engaged in a data communication session with an external communication system 100, in which case the algorithm 300 would deny or delay action on the charging request until the data communication session is over. Ultimately, if or when the algorithm 300 decides that charging can occur, the algorithm can cause the IMD 150 to transmit a confirmation to the charger 270 that charging can begin via RF link data 256. The external charger 270 may then automatically start to produce the magnetic charging field on MI power link 250 in response.

In the example of step 305, the charging request may at step 304 can come in the form of the charger 270 generating of the magnetic charging field via MI power link 250. In this example, the algorithm 300 detects this magnetic field and interprets it as a charging request. As noted earlier, the algorithm 300 can detect the presence of the magnetic charging field via charging sensor 238 as explained earlier. If charging is requested in this manner, it is not required that the IMD 150 send an acknowledgement back to the charger 270 that charging can begin, as the charger 270 is already providing the magnetic charging field.

Regardless of the manner in which charging is requested, the algorithm 300 can configured the IMD's circuitry to operate in a charging mode at step 306. The algorithm 300 can thus set X=0, to close or open the various switches to disable the telemetry circuity 223 and/or enable the charging circuitry 221. Charging of the IMD 150 thus commences (or continues) at step 306.

Step 308 monitors charging of the IMD 150 once the MI power link 250 is being generated. Such monitoring can occur through use of the battery monitoring circuitry 236, which can determine charging information such as Vbat, Ibat, and temperature for example, and/or the charging sensor 238, which determines whether the magnetic charging field is still present. As relevant here, monitoring at step 238 can include two inquiries: has the battery 222 been fully charged (e.g., has Vbat risen above a threshold Vt indicative that the battery is full); and is a MI power link 250 still being provided from the charger 270. Although not depicted in FIG. 6, one skilled in the art will understand that the IMD 150 can also telemeter charging information to the charger 270 via LSK data link 252 as necessary as the battery is being charged.

If the algorithm 300 determines that the battery 222 has been fully charged (e.g., Vbat>Vt), and the magnetic charging field is still being produced on MI power link 250 from the charger 270, the algorithm can move to step 310. At this step the algorithm 300 can cause the control circuitry 230 to transmit information to the charger 270 via LSK link 252 to disable further production of the magnetic charging field on MI power link 250. Such information may comprise a specific instruction to the charger to shut off the magnetic charging field, or may comprise information which allows the charger 270 itself to make its own determination whether the magnetic charging field should cease. For example, the information transmitted at step 310 may comprise the charging information discussed earlier (e.g., Vbat), with the charger 270 ceasing production of the magnetic charging field on MI power link 250 when Vbat reaches Vt. In either case, the algorithm at next step 312 can verify that the MI power link 250 has in fact ceased, which can again occur using either or both the battery monitoring circuitry 236 and/or the charging sensor 238.

At this point, the algorithm 300 knows that charging is no longer occurring (or needed), and can proceed back to step 302 to place the IMD again into the data communications mode (X=1) in preparation for potential future communications with external communication systems 100 and/or the charger 270.

The algorithm 300 can also configure the IMD 150 for data communications if at step 308 the algorithm 300 understands that a magnetic charging field is no longer being produced on MI power link 250. This might occur if the patient turns off or removes the charger from the vicinity of the IMD 150. In this case, the charger 270 would again need to instigate charging via a new charging request (steps 304, 305). To prevent from switching to data communications too quickly, e.g., in the event of only a temporary discontinuation of the magnetic charging field, the algorithm 300 may determine at that the MI power link 250 has been consistently inactive for some period of time (e.g., a few seconds) before returning back to the data communication mode at step 302.

Notice that algorithm 300 time multiplexes data communications and charging functionality, specifically determining when it is to operate in either mode, and configuring the IMD 160 appropriately to control operation of the antenna structure 200, the charging circuitry 221, and the telemetry circuitry 223. Given this method of control, filtering of received signals is not required because conflicts between data communications (at 2.4 GHz) and charging (e.g., at 13.56 MHz) should not occur. For example, it is not necessary to filter data received during charging, as the telemetry circuitry 223 can be disabled when the IMD is operating in the charging mode (X=0). Likewise, it is not necessary to filter a magnetic charging field received during data communications, as the charging circuitry 221 can be disabled when the IMD is operating in the data communications mode (X=1). In the example of FIG. 6, the time multiplexing algorithm 300 can operate solely within the IMD 150.

FIG. 7 shows another method by which algorithm 300 can operate, and in this example, the algorithm 300 can operate at least in part within the charger 270. In the example of FIG. 7, a charging session is time multiplexed into a number of charging periods of a duration D, with intervening telemetry periods provided to determine whether charging needs to continue or can end. The algorithm 300 as shown in FIG. 7 only relies upon RF data communications between the charger 270 and the IMD 150 along RF data link 256 to control and synchronize charging. Communications along LSK link 252 are not required.

Step 302 is as described earlier, with the algorithm 300 setting the IMD to the default data communications mode. Step 304 is also as described earlier, with the IMD 150 receiving a request from the charger 270 to begin charging via RF data link 256. As noted earlier, this request may be sent from the charger automatically without the user's knowledge, by turning on the charger for example. “Handshaking” would occur at this step. It is assumed in this example that the charger 270 cannot simply begin producing a magnetic charging field on MI link 250, with this field serving as a charging request (compare step 305, FIG. 6), although this could occur as well. Once the charging request has been received at the IMD 150, the algorithm 300 operating in the IMD 150 can determine whether charging can be enabled at the IMD 150, and hence whether the charger 270 can begin generating its magnetic charging field. As before, the algorithm 300 may reject or delay this request, if the IMD is busy with another task as described earlier. When the algorithm 300 decides that charging can occur, the algorithm can cause the IMD 150 to transmit a confirmation to the charger 270 that charging can begin via RF link data 256.

At step 318, the algorithm 300 operating in the IMD 150 initiates the charging mode (X=0), and the charger 270 may then automatically start to produce the magnetic charging field on MI power link 250. Such charging will occur for a set charging period duration D, which is preferably known by (e.g., programmed into) the algorithm 300 operating in both the IMD 150 and the charger 270 for proper synchronization. Duration D may comprise a period of time that would normally be too short to fully charge the IMD 150's battery 222. For example, duration D may comprise several seconds to several minutes.

Step 320 inquires whether duration D has been exceeded, and if not charging can continue. When duration D is exceeded, the IMD 150 and charger 270 again establish a data communication session at step 322 to exchange necessary charging information, and to determine whether charging should continue. More specifically, the charger 270 stops producing the magnetic charging field on power link 250, and the IMD defaults back to the data communications mode (X=1). After handshaking and establishing communications on RF data link 256, charging information may be transferred from the IMD 150 to the charger. For example, the IMD 150 can transmit the battery voltage Vbat to the charger, and may possibly transmit other relevant charging information as well, such as Ibat and temperature.

At step 324, one of the devices in the system—either the charger 270 or the IMD 150—determines whether further charging is required. If the charger 270 is to make this determination, the IMD 150 will need to have transmitted relevant charging information to the charger (e.g., Vbat, at step 322). If by contrast the IMD 150 is to make this determination, no further data may be transmitted to the charger 270. In any event, the device that makes this determination will assess the charging information (e.g., Vbat v. Vt) and determine whether further charging is required.

If further charging is not required (e.g., Vbat>Vt), the device making this determination can inform the other device of this decision in step 326, in effect allowing both devices to confirm or understand that further charging is not required. Such confirmation can occur using the RF data link 256 already established between the two devices. The current data communication between the charger 270 and the IMD 150 can then end, with the algorithm 300 returning to step 302 to allow the IMD 150 to establish other communication sessions in the future. Because the charger 270 has already ceased production of the magnetic charging field on MI power link 250 (step 322), the charger may simply turn off, or otherwise notify the user that charging is complete.

If further charging is required at step 324 (e.g., Vbat<Vt), the device making this determination can inform the other device of this decision in step 328, in effect allowing both devices to confirm and understand that further charging is required. In this circumstance, the data communications session can end, and the algorithm 300 can return to step 318. As discussed earlier, at this point the algorithm operating in the IMD 150 places it in the charging mode (X=0), and the algorithm operating in the charger 270 can again begin generating the magnetic charging field on MI power link 250. As before, this charging will occur for the duration D discussed earlier (step 320), with a new data communication established afterwards to determine and communicate whether further charging is required (322-328). Through this iterative process, further charging would eventually not be required (as Vbat increases during charging), and charging would cease.

The charging period durations D may be fixed during a charging session, or they may be varied as the algorithm 300 operates. For example, durations D may initially be longer, but may be shortened as charging progresses (i.e., as the battery becomes more charged). The extent to durations D might be adjusted by the algorithm 300 may depend on the charging information (e.g., Vbat, Ibat, temperature). For example, if the temperature is high, or if Vbat is approaching a full threshold (Vt), the durations D may be shortened. By contrast, the charging durations D might also be lengthened over time if desired.

To summarize, in the example of FIG. 7, the algorithm 300 operates in both the IMD 150 and the charger 270 to interleave telemetry periods (322-328) between each of the charging periods of duration D (318, 320). Note that by comparison to the charging period duration D, the interleaved telemetry periods would be quite short (tenths of a second). In this regard, the magnetic charging field is almost always on as the algorithm 300 of FIG. 7 operates, with the interleaved telemetry periods not significantly lengthening the time it would take to fully charge the IMD 150's battery 222.

While examples of time multiplexing algorithm 300 as shown so far set the IMD 150 to default to operation in the data communication mode (X=1), this is not strictly necessary, and instead the algorithm can default to configure the IMD 150 in the charging mode (X=0). This example is shown in FIG. 8. At step 350, the algorithm 300 configures the IMD 150 for operation in the charging mode by default (X=0). As such, data communications cannot (yet) occur on RF data links 254 and 256.

At step 352, the algorithm 300 inquires whether a charging request has been received. In this example, the charging request comprises the generation of the magnetic charging field on MI power link 250 from the charger 238, which can be sensed by the charging sensor 238. If a charging request has been received, and as shown in step 354, charging can commence and be monitored. This can occur in different manners as outlined earlier in FIGS. 6 and 7. For example, steps 308 to 312 of FIG. 6 can occur. Thus, charging can commence/continue, with the algorithm 300 monitoring whether the battery is fully charged, and/or whether the magnetic charging field on MI link 250 has ceased (308). When the battery is full, the IMD can transmit that fact to the charger 270 via LSK link 252 (310) so that the charger can turn off the charging field. Alternatively, steps 320 to 328 of FIG. 7 can occur, which as noted earlier interleaves charging periods on MI power link 250 with data communication periods on RF data link 256 to decide when charging should end. Afterwards (or if the magnetic charging field has ceased for some reason), the algorithm 300 can return to step 350, with the IMD 150 defaulting back to the charging mode (X=0).

If a charging request has not been received at step 352, the algorithm 300 can move to step 356 to inquire whether a delay X has been exceeded. This delay X is set to allow the IMD 150 to periodically configure itself for, and check the need for, data communications. Thus, when delay X is exceeded, and as shown in step 358, the algorithm 300 places the IMD 150 in a data communications mode (X=1). Thus, and as described earlier, the IMD 150 can start advertising its presence on RF data links 254 and 256 and see whether a data communications request is issued from an external communication system 100 or the charger 270 (step 360). If no communication request is received, the algorithm can inquire whether a delay period Y has been exceeded (362). Delay period Y determines how long the IMD 150 will remain in the data communications mode and check for the need for RF data communications with external devices. If delay period Y expires without a data communications request, the algorithm can return to step 350, once again setting the IMD 150 in the charging mode (X=0). It should be noted that delay periods X (356) and Y (362) are a matter of design choice, with values for each depending on whether it is more important to allow charging to quickly occur when requested (in which case period X would relatively long compared to period Y), or whether to allow data communications to quickly occur when requested (vice versa).

If a communications request is received at step 360 (and after handshaking), the algorithm 300 may inquire whether this request comprises a charging request from the external charger 270 as received on data link 356. This step isn't required, because the charger 270 as noted earlier can request charging simply by turning on its magnetic charging field (at step 352). Nevertheless, the charger 270 may also request charging through RF data link 356 as well. Should this occur, the algorithm 300 can configure the IMD for the charging mode (X=0) and proceed to step 354 to charge and monitor the IMD 150. If the communications request is not a charging request at step 364, it may be a request from an external communication system 100 on RF data link 254 (or some other form of communication request from the charger 270 on RF data link 256). In this circumstance, the devices have their communication session at step 368 (on either of RF data links 254 or 252), until this session is ended and complete. At that point, the algorithm can proceed back to step 350 to set the (default) charging mode (X=0).

While it can be beneficial to time multiplex usage of the antenna structure 200, using algorithms 300 for example, such time multiplexing is not strictly required, and instead charging and data communications can occur simultaneously, without the need to affirmatively enable/disable the telemetry and charging circuitries 221/223. Higher-frequency data communications received at or transmitted form the antenna structure 200 will not be hindered by parasitic effects of the charging circuitry 221's rectifier circuitry 216 (FIG. 4), because the RF feed 202 is not provided directly at connections 204a or 204b. This prevents high frequency signals at connection 202 from shorting to ground at the rectifier 216 (i.e., through capacitances inherent in the rectifier). Moreover, the interface circuitry 226 within the telemetry circuitry 223 can be designed to filter the lower-frequency charging signals received at the antenna structure 200.

Various modifications to the antenna structure 200 are possible, and are shown in subsequent figures. One skilled in the art will recognize that the various modifications as shown can be combined in different manners. Not all of these combinations are explicitly shown.

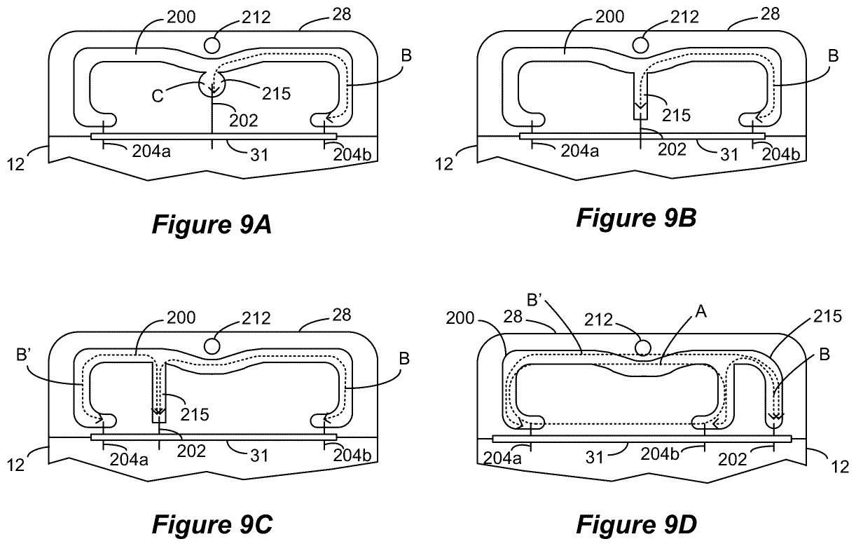

In FIG. 9A, the antenna structure 200 includes an extension 215 to which the RF feed 202 is connected. This extension 215 can operate as a patch antenna. This patch has an area C as shown, and can be dimensioned to promote efficient data communication at the frequencies used for data communications on RF data links 254/256. In the example shown, extension 215 is largely circular in area, although this isn't strictly necessary and other shapes (rectangles, square, etc.) are possible. Although not shown, extension 215 can also include slots, and operate at least in part as a slot antenna. Note that the addition of extension 215 to the antenna structure 200 may change the length B (FIG. 3C) of the monopole antennas formed when RF feed 202 is active, which may be useful to tuning the antenna structure 200 to operate at the frequencies involved (e.g., Bluetooth). Extension 215 is largely implicated during the data communications mode; it is not implicated in the charging mode when the antenna structure 200 is induced with a current by the magnetic charging field (MI link 250) between connections 204a and 204b. This is because extension 215 is not significantly within the current path formed between connections 204a and 204b as shown. In the example shown in FIG. 9A, the extension 215 is planar with the rest of the antenna structure 200 and may be formed out of the same sheet of conductive material as the rest of the antenna structure.

FIG. 9B also shows the use of an extension 215, although in this case the extension is largely linear, without a substantial area C. Extension 215 nevertheless still affects the lengths B of the monopole antennas formed, which as just noted can be useful in tuning the antenna. In FIG. 9B, the extension 215 is centered and connects to the middle of the antenna structure 200 (e.g., top portion 200a) between the ends where connections 204a and 204b are connected. In FIG. 9C, the extension 215 is not centered in the antenna structure 200, which forms different lengths for the two monopole antennas—length B between connection 202 and 204a and length B′ between connection 202 and 204a. Having antennas with differing lengths can be useful to increase the bandwidth or efficiency with which the antenna structure 200 operates in the data communications mode.

In FIG. 9D, the extension 215 is not located within the area A defined by the loop between connections 204a and 204b, which may reduce interference when the antenna structure 200 is receiving a magnetic charging field via MI power link 250. This configuration also forms monopole antennas with different lengths B and B′ as shown.

In the examples shown in FIGS. 9A-9D, the extension 215 is planar with the rest of the antenna structure 200, and may be formed of the same material. The various examples of extension 215 may also be made of different materials, and connected to the general loop shape of the antenna structure 200.

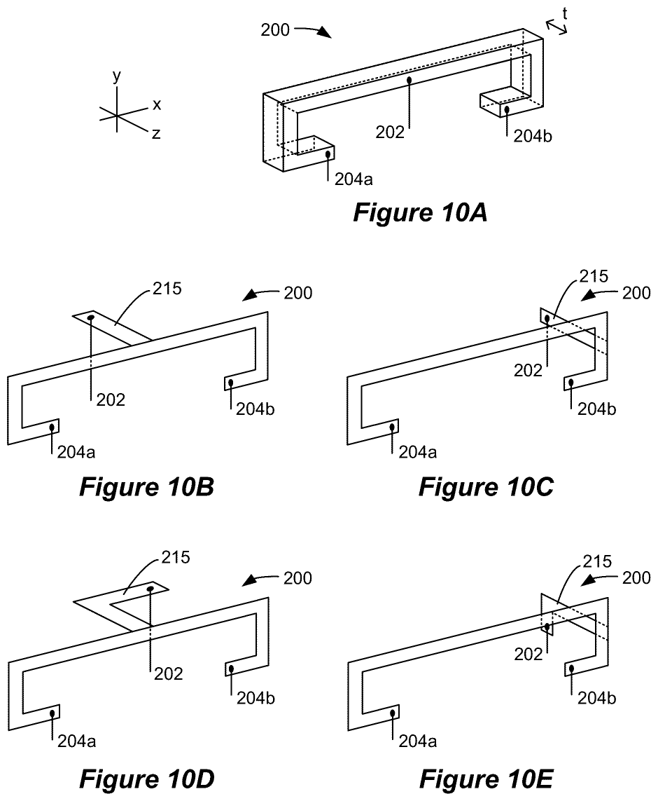

Efficiency of data communications in particular may be further improved by providing some three dimensionality to the antenna structure 200, and the examples of FIGS. 10A-10E show various possible three-dimensional structures for the antenna structure 200. In these examples, the general loop shape of the antenna structure 200 is still planar (in the x-y plane), and thus power reception during the charging mode is unaffected. But the antenna structure 200 also has significant length in the z direction. For example, in FIG. 10A the antenna structure 200 is formed with a substantial thickness t (in direction z). This may occur by forming the antenna structure 200 using a particularly thick conductive sheet, or by forming the antenna structure 200 as several thinner layers.

In FIGS. 10B-10E, the antenna structures 200 are made three dimensional by including extension 215 at different locations having a substantial projection in the z direction. As in earlier examples, these extensions 215 are connected to RF feed 202. However, this is not strictly necessary. Although not shown, the extensions 215 could also be connected to connections 204a or 204b, with RF feed 202 connected instead at one of the ends of the loop (in place of 204a or 204b). That is, the three connection points to the antenna structure 200 can be varied between 202, 204a, and 204b in accordance with user preferences. The extensions 215 as shown in these figures can be formed by bending the conductive material for the antenna structure 200, although as mentioned earlier, these extensions can also be made from separate materials and affixed to the antenna structure as well.

Examples of the antenna structures 200 and related circuitry shown so far assume that three connections are made to the antenna structure 200—connections 204a and 204b at the ends of the loop coupled to the charging circuitry 221, and an RF feed 202 coupled to the telemetry circuitry 223. However, this is not strictly required, and instead there may only be two connections to the antenna structure 200, because one of the end-of-loop connections can be used as a RF feed as well.

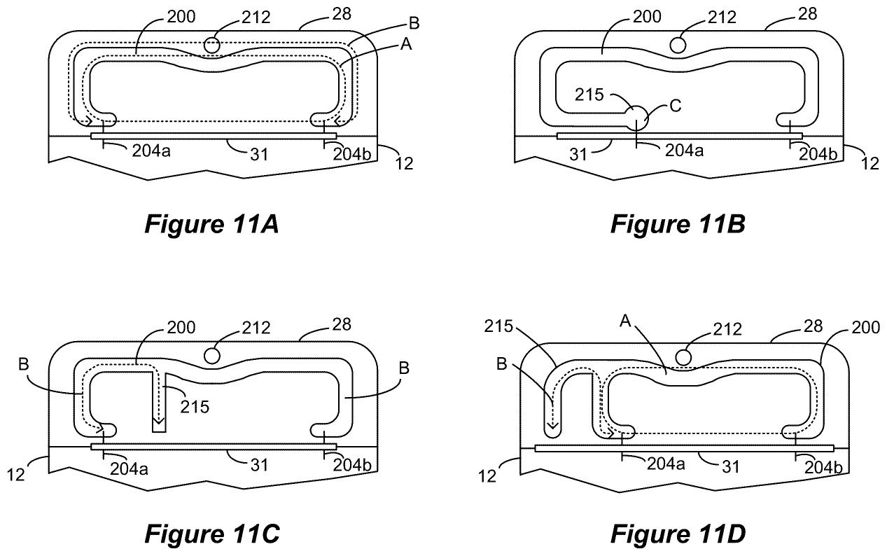

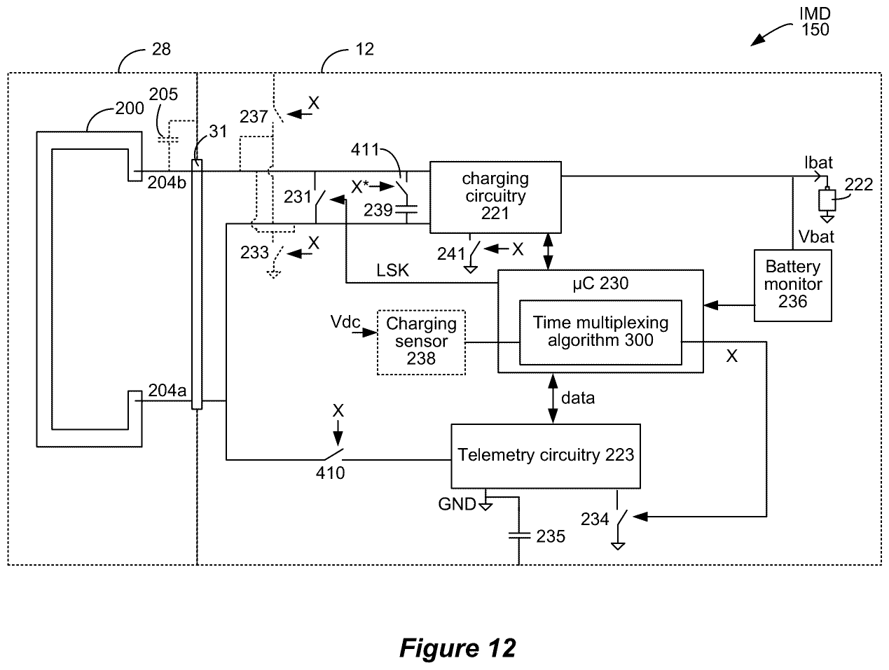

An antenna structure 200 capable of operation in data communication and charging modes, but having only two connection points, is first shown in FIG. 11A, with modifications to the IMD circuitry shown in FIG. 12. Only connections 204a and 204b at the ends of the antenna structure 200 are present. These connections 204a and 204b, as in earlier examples, are connected to the charging circuitry 221 and act as described earlier in the charging mode (X=0) to capture magnetic flux provided by MI power link 250 to provide power to the IMD 150 through magnetic induction.

Unlike earlier examples, RF feed connection 202 is absent, and instead connection 204a at the end of the antenna structure 200 selectively acts as the RF feed, and is coupled to the telemetry circuitry 223 and active in the data communications mode (X=1). As before, switch 234 can be used to enable the telemetry circuitry 223 and activate this RF feed. To prevent the high frequency data signals on the antenna structure from shorting through the resonant capacitor 239, a switch 411 can be added in series with this capacitor, which is opened in the data communication mode, in effect disabling the charging circuitry 221 (in addition to, or in place of, switch 241). Switch 411 can be controlled with the inverse of control signal X, i.e., X*. An optional switch 410 intervening between the telemetry circuitry 223 and connection 204a can also be closed in the data communication mode, although this switch 410 is not required if the telemetry circuitry 223 is enabled and disabled using switch 234. When in the data communication mode, the antenna structure 200 is established as a single monopole antenna of length B (essentially the entire length of antenna structure in FIG. 11A) which is fed at connection 204a and parasitically grounded at connection 204b. As explained earlier, this grounding (to the case 12 acting as a ground plane) is affected by capacitances 205 and 235.

During the charging mode (X=0), switch 411 is closed to connect the resonant capacitor 239 to the antenna structure 200. Switch 410, if present, may also be opened to disable the telemetry circuitry 223, allowing connection 204a to float as is preferable during receipt of the magnetic charging field and the build up of an AC voltage across connections 204a and 204b. Other switches described earlier (e.g., 234, 241, 233, 237, 233) may as before be used to effectively enable and disable the telemetry circuitry 223 and charging circuitry 221 in the respective operating modes as set by the time multiplexing algorithm 300 (X). Time multiplexing algorithm 300 can otherwise operate in any of the manners described earlier (see FIGS. 6-8).

Modifications are possible to the two-connection-point example of the antenna structure 200. For example, FIG. 11B shows use of an extension 215, similar to that discussed earlier with respect to FIG. 9A, the details of which are not repeated here. In the example shown, the extension 215 is located at the connection 204a, although this is not strictly necessary, and instead extension 215 can be located at other points along the length of the antenna structure 200 (including at connection 204b). FIG. 11C shows use of an extension 215 at an intermediate point along the length of the antenna structure 200. Notice that this may change (shorten) the effective length B of the monopole antenna, which may be parasitically grounded to the case 12 at the end of the extension 215. In FIG. 11D, and similar to FIG. 9D discussed earlier, the extension 215 is not located within the area A defined by the loop between connections 204a and 204b, which may reduce interference when the antenna structure 200 is receiving a magnetic charging field via MI power link 250. This may also change (shorten) the effective length B of the monopole antenna, which again may be parasitically grounded to the case 12 at the end of the extension 215.

The examples of FIGS. 11A-11D all show the antenna structure 200 as planar. However, and although not shown, modifications discussed earlier (see FIGS. 10A-10E) can also be employed to render these antenna structures three-dimensional, which as noted earlier may improve performance in the data communications mode in particular.

Although disclosed as a structure that can provide both RF data communications and charging functionality, the disclosed examples of the antenna structure 200 can also be used in an IMD to provide just one of these functions individually, i.e., to provide just RF data communications or to provide just charging functionality. That is, it is not required that the antenna structure 200 necessarily implements both of these functions in an IMD.

Although examples of the present invention are described as being used in implantable stimulation devices system such as a spinal cord stimulation system, as indicated above, the antennas, circuitries, and algorithms described herein may be used in other implantable medical device systems. For example, in some implementations, the implantable medical device may include an electric pump that is configured to move fluid within the implantable medical device to inflate or deflate an inflatable member. For example, in some implementations, the implantable medical device may be an inflatable penile implant. In some cases, inflatable penile implants arc used to help address erectile disfunction issues.

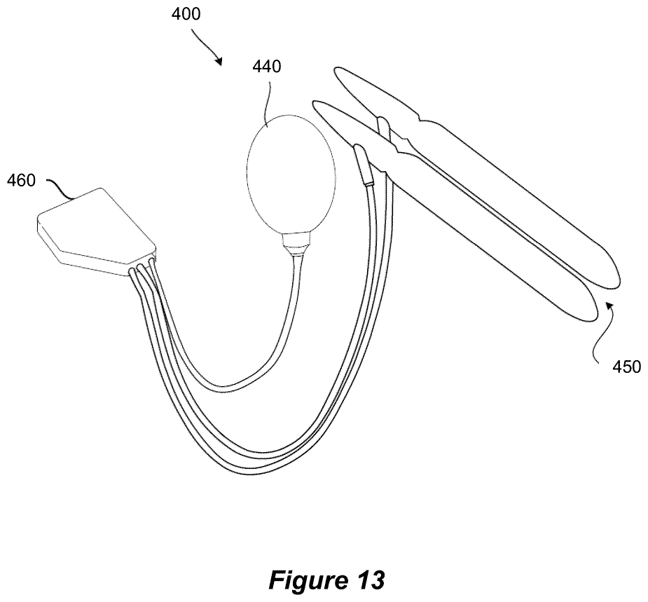

As illustrated in FIG. 13, an inflatable penile implant 400 (a type of implantable medical device, IMD) includes a fluid reservoir 440 configured to be placed within a pelvic region of a patient and one or more inflatable members 450 configured to be placed within a penis of the patient. The inflatable penile implant 400 also includes a housing 460 (including a case and a header) that is operatively coupled to the fluid reservoir 440 and to the inflatable member(s) 450. The housing 460 may house a power source (such as a rechargeable battery) and a pump or pumps (such as a piezo-electric pump or pumps) configured to move fluid to and from the inflatable member(s) 450 to place them in an inflated configuration or a deflated configuration. The housing 440 may also house antennas and circuitry, such as antennas and circuitry as described herein, to allow the inflatable penile implant 400 to be controlled from an external communication system 100 disposed outside of the body of the patient, and to provide the ability for the power source of the inflatable penile implant 400 to be recharged by external charger 270.

As discussed earlier, the various algorithms described herein (e.g., 300, sec FIGS. 6-8) can be implemented as firmware or software, and such instructions may be embodied in a non-transitory computer readable media, such as a solid-state memory (e.g., control circuitry 230 in the IMD 150 and/or 272 in the charger 270), optical or magnetic disks, and the like. These media may be within the IMD 150, charger 270, or in external systems in manners downloadable to the IMD 150 and/or charger 270, such as on various Internet servers (e.g., 86, FIG. 2), portable or stationary disks, manufacturing computer systems, and the like.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An implantable medical device (IMD), comprising:
a case;
a non-conductive header attached to the case;
an antenna within or on the header, wherein the antenna is configurable to operate in a first mode for far-field radiofrequency (RF) data communications with an external system, and in a second mode to receive a near-field magnetic charging field to power the IMD; and
control circuitry within the case configured to configure operation of the antenna in the first and second modes in a time multiplexed manner.

2. The IMD of claim 1, wherein the antenna is within the header.

3. The IMD of claim 2, wherein the header is overmolded over the antenna.

4. The IMD of claim 1, wherein the header further comprises at least one lead connector into which at least one lead can be inserted.

5. The IMD of claim 4, wherein the antenna is formed in a plane.

6. The IMD of claim 5, wherein the plane is perpendicular to a top of the case to which the header is attached.

7. The IMD of claim 6, wherein the plane is offset in the header in a direction parallel with the top of the case.

8. The IMD of claim 5, wherein the antenna comprises at least one planar sheet of metal.

9. The IMD of claim 1, wherein the antenna is three dimensional.

10. The IMD of claim 1, wherein the antenna is loop shaped.

11. The IMD of claim 1, wherein the antenna comprises a top horizontal portion, a right vertical portion, and a left vertical portion.

12. The IMD of claim 1, further comprising a feedthrough between the header and the case, and further comprising a plurality of electrical connections to the antenna, wherein the plurality of electrical connection pass through the feedthrough.

13. The IMD of claim 12, further comprising a resonant capacitor, wherein an inductance between the first and second ends and the resonant capacitor comprise a resonant tank to form an AC voltage across the first and second ends in the second mode in response to the magnetic charging field, wherein the AC voltage provides the power to the IMD.

14. The IMD of claim 13, wherein during the first mode the control circuitry configures a third of the electrical connections to operate as an RF feed for the RF data communications.

15. The IMD of claim 14, wherein none of the first, second, or third electrical connections are directly connected to the case in either of the first or second modes.

16. The IMD of claim 14, wherein during the second mode the third electrical connection is inactive.

17. The IMD of claim 1, wherein the control circuitry is configured to default to operation in the first mode.

18. The IMD of claim 17, wherein the control circuitry is configured to receive a charging request to switch to operation in the second mode.

19. The IMD of claim 1, wherein the control circuitry is configured to default to operation in the second mode.

20. The IMD of claim 19, wherein the control circuitry is configured to automatically switch operation to the first mode after a duration.
